(19) United States
(12) Patent Application Publication
(10) Pub. No.: US 2008/0240980 A1
(43) Pub. Date: Oct. 2, 2008

(54) SUPERSONIC BACTERICIDAL METHOD CONTAINING CHITOSAN AND ITS FACILITIES

(76) Inventors: CHUAN-PAN HUANG, TAINAN CITY (TW); CHEN-LUNG HUANG, TAINAN CITY (TW)

Correspondence Address: J C PATENTS, INC., 4 VENTURE, SUITE 250, IRVINE, CA, 92618, US

(21) Appl. No.: 11/694,586

(22) Filed: Mar. 30, 2007

(51) Int. Cl. A61L 2/18

(52) U.S. Cl. 422/28; 422/20; 422/128

(57) ABSTRACT

The invention relates to a supersonic bactericidal method and facilities containing chitosan, consisting of a bactericidal liquid and a supersonic atomizer. The bactericidal liquid is composed of water (X) and chitosan (Y), with a preferable ratio, X: Y=500:1. However, other ratios are available, too. The supersonic atomizer is preferably operated under a catalysis frequency between 2.4˜3.6 MHZ. So, when the bactericidal liquid is atomized through the supersonic atomizer, it is to be atomized having a diameter ranging from 300 nm to 150 nm, experimentally approved to have a significant bactericidal effect and be able to relatively save energy.

### BACKGROUND OF THE INVENTION

1. Field of the Invention

This invention relates to a supersonic bactericidal method containing chitosan and its facilities, particularly to one having a bactericidal liquid composed of water (X) and chitosan (Y) with a ratio (X):(Y)=500:1, and an atomizer able to atomize the bactericidal liquid into drops with a diameter ranging from 300 nm to 150 nm, which are spread into an indoor or certain space to sterilize.

2. Description of the Prior Art

A conventional atomized bactericidal composition, as revealed in Taiwan Patent No. 200538468, an atomized bactericidal composition and its method of preparation, consists of chitosan, degraded by hydrogen peroxide to have a molecular weight ranging from 10000˜100000, and ethanol. In preparation, the chitosan is added in an alkaline solution and then heated. Next, hydrogen peroxide is added in to keep the chitosan degraded to have a molecular weight ranging between 10000˜100000. Then, the degraded chitosan is dissolved by an acid to filter out the insoluble. Alkaline titration is used to keep the chitosan precipitated. The precipitated chitosan is further cracked into powder, which is successively blown with carbon dioxide so as to dissolve the chitosan. Finally, ethyl alcohol is added into the solution to become the conventional bactericidal composition that can achieve a bactericidal effect as high as 99.9% and keep the sprinkled locations remained not sticky.

That is, the characteristic of the conventional bactericidal composition is to keep it from remained sticky after atomized. As described above, in preparation of the conventional bactericidal mixture, first the chitosan is added in an alkaline solution and then heated. Next, hydrogen peroxide is added in to keep the chitosan degraded to have a molecular weight ranging between 10000˜100000. Then, the degraded chitosan is dissolved by an acid to filter out the insoluble. Then alkaline titration is used to keep the chitosan precipitated. The precipitated chitosan is further cracked into powder, which is successively blown with carbon dioxide so as to dissolve the chitosan. Finally, ethyl alcohol is added into the solution to become the conventional bactericidal composition. In using, the conventional bactericidal composition is sprayed on a location expected to be sterilized.

### SUMMARY OF THE INVENTION

The objective of this invention is to offer a supersonic bactericidal method containing chitosan and its facilities.

The main components of the invention are a bactericidal liquid and a supersonic atomizer. The bactericidal liquid is a mixture of water (X) and chitosan (Y) with a ratio (X):(Y)=500:1, catalyzed by the supersonic atomizer with a catalysis frequency of 2.4 MHZ˜3.6 MHZ to become drops with a diameter ranging from 300 nm to 150 nm, which are spread into an indoor or certain space to sterilize.

The main characteristics of the invention are described below.

1. The bactericidal liquid has a preferable mixing ratio, 500:1, for water (X) and chitosan (Y) because it contains optimum chitosan after atomized.

2. The water (X) can be altered to be larger or smaller than 500 and the chitosan (Y) can be altered to be larger or smaller than 1, in accordance with diverse requirements, although the ratio of the X: Y=500:1 is most preferable.

3. The supersonic atomizer is preferably operated under a catalysis frequency ranging between 2.4 MHZ and 3.6 MHZ.

4. The bactericidal liquid is to become mists with a diameter ranging from 300 nm to 150 nm after atomized by the supersonic atomizer.

5. The facilities of the supersonic bactericidal method containing chitosan includes a water reservoir, enabling the bactericidal liquid atomized via the supersonic atomizer to be dispersed into mists with diameters ranging from 300 nm to 150 nm.

### BRIEF DESCRIPTION OF DRAWINGS

This invention is better understood by referring to the accompanying drawings, wherein:

FIG. 1 is a flow chart of a preferred embodiment of a supersonic bactericidal method containing chitosan in the present invention;

FIG. 2 is a first preferred embodiment of facilities of the supersonic bactericidal method containing chitosan in the present invention; and

FIG. 3 is a second preferred embodiment of facilities of the supersonic bactericidal method containing chitosan in the present invention.

### DETAILED DESCRIPTION OF THE PREFERRED EMBODIMENT

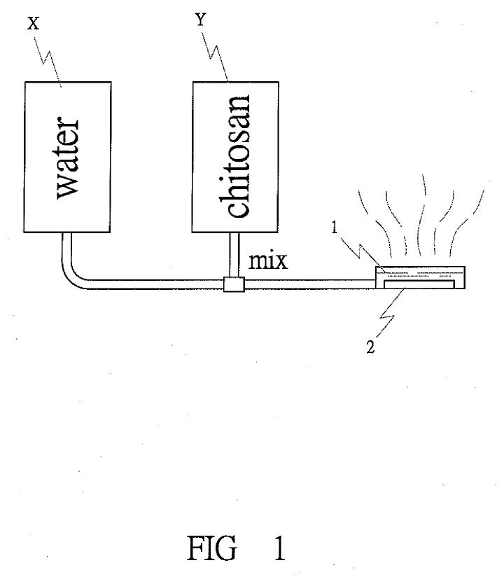

As shown in FIG. 1, a preferred embodiment of a supersonic bactericidal method containing chitosan in the present invention including a bactericidal liquid 1 and a supersonic atomizer 2.

The bactericidal liquid 1 is a mixture of water (X) and chitosan (Y). The preferable mixing ratio of water (X) and chitosan (Y) is 500:1 because each liquid drop atomized with the bactericidal liquid 1 contains an optimum quantity of bactericidal chitosan according to experiments. Of course, the X can be altered to be larger or smaller than 500, or the Y can be altered to be larger or smaller than 1, for diverse bactericidal requirements.

The supersonic atomizer 2 is preferably operated under a catalysis frequency between 2.4˜3.6 MHZ, which is able to atomize the bactericidal liquid 1 into drops with a diameter ranging from 300 nm to 150 nm. Via facilities of the supersonic bactericidal method containing chitosan, the atomized drops are transferred or spread into an indoor or a targeted space so as to destroy bacteria existing in the space, providing a safer and more comfortable living space.

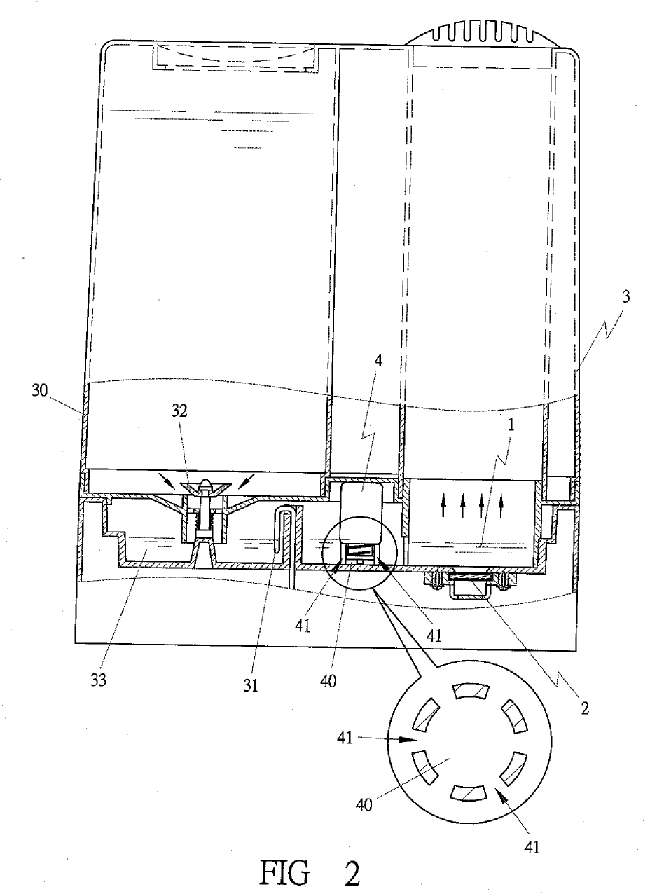

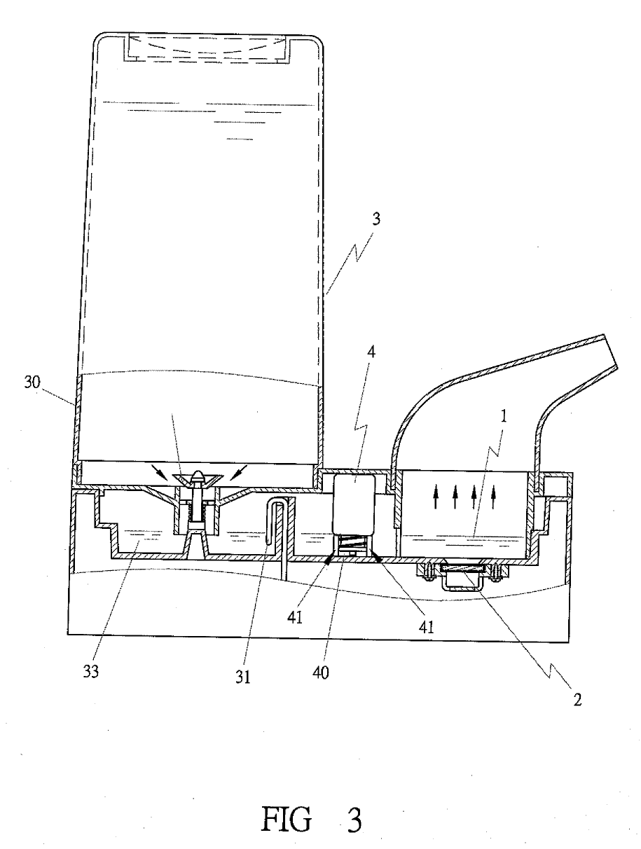

As shown in FIGS. 2 and 3, a preferred embodiment of facilities of the supersonic bactericidal method containing chitosan in the present invention includes a main body 3 and a chitosan tank 4.

The main body 3 is provided with a water reservoir 30 and a water level detector 31 installed in a water tank 33 separately installed just under the bottom of the water reservoir 30 for detecting water level in the water tank 33 to remind a user to refill water in the water reservoir in time to keep the facilities operating normally. In addition, a water valve 32 is set at the bottom of the water reservoir 30 to be controlled by the water level of the water tank 33 so that the water in the water reservoir 30 may drop therethrough duly into the water tank 33.

The chitosan tank 4 installed beside the water tank 33 is provided with a tank base 40 and plural exits 41 set around the tank base 40 for the chitosan therein to flow out and blend with the water coming from the water tank 33 to form the bactericidal liquid 1.

The supersonic atomizer 2 is installed under one side of the bottom of the main body 3, able to atomize the bactericidal liquid 1 properly. As mentioned previously, the supersonic atomizer 2 is preferably operated under a catalysis frequency between 2.4˜3.6 MHZ, so that the bactericidal liquid 1 can be atomized into tiny drops with a diameter ranging from 300 nm to 150 nm. And, via the facilities of the supersonic bactericidal method containing chitosan, the atomized drops are transferred or spread into an indoor or a targeted space so as to destroy bacteria existing in the space, providing a safer and more comfortable living space.

Moreover, the bactericidal liquid 1 of the invention has been proved containing chitosan by United States Environmental Protection Agency. And, according to tests done by SGS Taiwan Ltd. and Guangzhou Industry Microbe Test Center, an one-hour test of spreading the atomized bactericidal liquid 1 into an indoor space has proved that it can diminish 66.67% of natural bacteria and 44.5% of staphylococcus, and an eight-hour test has proved that it can get rid of 99.99% of bacteria, and 42% of ammonia and 65% of formaldehyde of organic solvents. Obviously, the bactericidal liquid 1 of the invention is practically effective for sterilization.

While the preferred embodiment of the invention has been described above, it will be recognized and understood that various modifications may be made therein and the appended claims are intended to cover all such modifications that may fall within the spirit and scope of the invention.

What is claimed is:

1. A supersonic bactericidal method containing chitosan comprising:
a bactericidal liquid composed of water (X) and chitosan (Y) and having a mixing ratio, X/Y;
a supersonic atomizer operated under a catalysis frequency between 2.4˜3.6 MHZ; and
said supersonic atomizer atomizing said bactericidal liquid into drops with a diameter ranging from 300 nm to 150 nm.

2. A supersonic bactericidal method containing chitosan as claimed in claim 1, wherein the mixing ratio of the water and the chitosan is: X/Y=500/1.

3. A supersonic bactericidal method containing chitosan as claimed in claim 1, wherein the mixing ratio, X/Y, is larger than 500/1.

4. A supersonic bactericidal method containing chitosan as claimed in claim 1, wherein the mixing ratio, X/Y, is smaller than 500/1.

5. Facilities of a supersonic bactericidal method containing chitosan comprising:
a main body provided with a water reservoir, a water tank installed just under said water reservoir, a water level detector for detecting water level in said water tank, and a water valve provided at a bottom of said water reservoir and used to control the water level in said water tank; and
a chitosan tank installed beside said water tank and provided with a tank base and plural exits set around said tank base for said chitosan in said chitosan tank to flow out therethrough and blend with water coming from said water tank to form said bactericidal liquid.

6. Facilities of a supersonic bactericidal method containing chitosan as claimed in claim 5, wherein said water level detector is a magnetic element.

7. A supersonic bactericidal method containing chitosan as claimed in claim 5, wherein said supersonic atomizer is preferably operated under a catalysis frequency between 2.4˜3.6 MHZ.
